(19) United States
(12) Patent Application Publication
(10) Pub. No.: US 2002/0198172 A1
(43) Pub. Date: Dec. 26, 2002

(54) Method of treating motor neuron diseases and demyelinating diseases with citicoline

(76) Inventors: Bobby W. Sandage JR., Lexington, MA (US); Glenn L. Cooper, Sudbury, MA (US); Kenneth W. Locke, Littleton, MA (US); Takashi Kiyoizumi, Wellesley, MA (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/207,111

(22) Filed: Jul. 30, 2002

Related U.S. Application Data
(63) Continuation of application No. 09/129,778, filed on Aug. 6, 1998, now abandoned.
(60) Provisional application No. 60/067,017, filed on Dec. 1, 1997.

(51) Int. Cl. A61K 31/7072

(52) U.S. Cl. 514/49

(57) ABSTRACT

The invention is directed to a method of treating a motor neuron disease or demyelinating disease, by the administration of citicoline to patients with a motor neuron disease or a demyelinating disease. The method is useful in the treatment of ALS and MS and maximizes the chances for a reduction, alleviation, or amelioration of ALS or MS symptoms in a patient. Combination treatment regimens are also disclosed along with compositions for use therewith.

### FIELD OF THE INVENTION

The present invention relates to a method of treating motor neuron and demyelinating diseases, including amyotrophic lateral sclerosis (ALS) and multiple sclerosis (MS). In particular, the invention relates to the use of citicoline (cytidine-5′-diphosphocholine or CDP-choline) in the treatment of these diseases.

### BACKGROUND OF THE INVENTION

There are a large number of identifiable motor neuron diseases and demyelinating diseases. The most common diseases of these types are ALS and MS, respectively.

ALS, also known as Lou Gehrig's disease, is a progressive disease of the nervous system marked by muscular weakness and atrophy with spasticity and hyper-reflexia due to degeneration of motor neurons of the spinal cord, medulla and cortex of the brain.

Motor neurons are classified as either myelinated or non-myelinated and their membranes are comprised of mainly neuronal lipids, sphingomyelin, phosphatidylcholine, phosphatidylethanolamine, and phosphatidylserine, each of which may contain varying amounts of acyl phospholipids. ALS is characterized by a progressive loss of upper and lower motor neurons, which includes the loss of the lipid membranes.

Motor neurons are among the largest of all nerve cells in the brain and spinal cord, and function to send messages to muscles throughout the body. In ALS, motor neurons die and the muscles do not receive these messages. As a result, muscles weaken as they lose their ability to move. Eventually, most muscle action is affected, including that which controls swallowing and breathing, as well as the movement of major muscles in the arms, legs, back and neck. There is, however, no loss of sensory nerves, so patients with ALS retain their sense of feeling, sight, hearing, smell and taste. Their mental capacity also remains relatively undiminished, and hence ALS patients remain fully cognizant. While the course of ALS is extremely variable and it is difficult to predict the rate of disease progression, the majority of patients with ALS progressively weaken and die over a three-to-five year period.

The first signs of ALS are often arm and leg weakness, muscle wasting and faint muscle rippling. These symptoms occur because muscles are no longer receiving the nutrient signals they need for growth and maintenance—a result of motor neurons dying. ALS nerve degeneration may also cause muscle cramps and vague pains, or problems with speech and swallowing.

ALS occurs in two forms. In hereditary or familial ALS, a defective gene is passed to successive generations. This accounts for about 10% of reported cases. The remaining 90% of cases are of unknown etiology.

Recent research on inherited ALS has led to the discovery of a defective gene believed to affect an enzyme called superoxide dismutase. This enzyme rids the body of free radicals, which, if not eliminated, can cause nerve cells to die. Free radicals are also associated with a number of other diseases, and are even implicated in the aging process itself.

Recently available treatments for ALS include medications which increase survival time and/or aid quality of life by maintaining muscle function. Examples of such medications include various nerve growth factors. While these treatments represent an incremental therapeutic advance, a cure for ALS remains elusive. Thus, the primary treatment of ALS still focuses on managing symptoms with physical, occupational, speech, respiratory and nutritional therapy. For instance, drugs and the application of heat or whirlpool therapy may help to relieve muscle cramping. Moderate exercise can help maintain muscle strength and function.

Drugs have also been used to treat fatigue, but may worsen muscle cramps. GDNF, or Glial Cell-Derived Neurotrophic factor, has been investigated in ALS, and is administered directly into the brain. The drug had been shown to promote the growth of nerve cells in animals. Pain in the limbs is common, persistent and hard to control. Nonsteroidal anti-inflammatory drugs and simple analgesics may bring relief. When pain is a major problem, opiates are used in doses that are effective in relieving the pain.

Spasticity is a relatively minor problem which is caused by a lower motor neuron lesion reducing the number of functioning motor units to such a degree that the muscles are more hypo- than hypertonic. Baclofen is the drug of choice for muscle spasms. Riluzole and is often also used to treat ALS.

Other motor neuron disorders exhibit symptoms similar to ALS but may have one or more recognizable differences. Kennedy's disease (X-linked spinobulbar muscular atrophy) is an X-linked lower motor neuron disease characterized by progressive muscular atrophy usually beginning in mid-adult life. This disease is distinguished from ALS by the absence of hyperreflexia and spasticity.

Adult Tay Sach's disease is caused by Hexosaminidase enzyme mutations that produce lower motor neuronopathies that closely mimic ALS. The disease is slowly progressive and may include dysarthria and cerebellar atrophy. Spasticity may also be present, but is rare.

Spinal muscular atrophy (SMA), is a group of familial disorders which affect large lower motor neurons. Muscle tissue often exhibits evidence of denervation atrophy. Infantile SMA (SMA I, Werdnig-Hoffman disease) is rapidly fatal, death generally ensuing within the first year of life. Chronic childhood SMA (SMA II) progresses slowly, beginning in childhood. Juvenille SMA (SMA III, Wohlfart-Kugelberg-Welander disease) generally has a late childhood or early adolescence onset and runs a slow course.

Primary lateral sclerosis (PLS) is rare disorder arising sporadically in adults from mid- to late-life. Symptoms include progressive spastic weakness of the limbs with spastic dysarthria and dysphagia. Fasciculations, amyotrophy and sensory changes are absent.

Familial spastic paraplegia (FSP) is a hereditary disease characterized by progressive spastic weakness which begins in the distal lower extremities.

Progressive neural muscular atrophy is a collection of degenerative disorders characterized by progressive weakness and wasting of skeletal muscles combined with sensory changes. The most common example is Charcot-Marie-Tooth (CMT) disease. This and many other progressive neuromuscular atrophy diseases are hereditary.

Various syndromes of progressive visual loss may also be attributed to neurodegenerative disorders. Examples include various forms of Friedreich's ataxia which are characterized by a slowing of conduction in the optic nerves. There are two broad categories of visual pathology, namely, selective degeneration of retinal ganglion cells with secondary optic atrophy and a more diffuse degeneration involving all retinal components. An example of the latter is retinitis pigmentosa.

Of the various demyelinating diseases, MS is by far the most well known. The causative agent of MS is unknown, although both infectious agents and autoimmunity are suspected. MS often strikes in early adulthood and is characterized by the formation of lesions (demyelinated plaques) in the central nervous system. The afflicted individual can exhibit lack of coordination, dysarthria (slurred speech), numbness, paralysis and/or urinary incontinence. Blindness has also been reported. Unlike the linear progression of ALS, however, the course of MS often involves spontaneous remission followed by relapses. Fortunately, in a majority of cases, permanent remission eventually occurs, but only after successively less severe relapses, and with an average duration of illness spanning 27 years.

Despite the fact that many MS patients experience eventual remission, the long life span of the disease inflicts psychological and economic hardship on patient and caregiver alike. Hence, treatment of at least the symptoms of the disease is highly advantageous.

Another demyelinating disease is acute disseminated encephalomyelitis (ADEM). This disease is distinguished from MS by having a monophasic course. It is often associated with immunization (postvaccinal encephalomyelitis) or infection (postinfectious encephalomyelitis). Symptoms include widely scattered small foci of perivenular inflammation and demyelination and may be chronic in nature.

Acute hemorrhagic leukoencephalitis (AHL) is characterized by perivenous demyelination and intense infiltration by mononuclear and polymorphonuclear inflammatory cells. The clinical course resembles severe forms of ADEM, but may be even more explosive in onset and progression. Death may occur within two to four days of onset, although complete neurologic recovery has been observed. Occasionally, as with ADEM, the disease may take a chronic course similar to MS.

Although there are some treatments for these diseases, there remains a need in the art for a method of treating motor neuron and demyelinating diseases, such as ALS and MS, which provides for both increased relief of symptoms and at least temporary cessation or even reversal of neuronal damage.

### SUMMARY OF THE INVENTION

The present invention meets this need by providing a method for treating motor neuron and demyelinating diseases which comprises administering an effective amount of citicoline or a pharmaceutically-acceptable salt thereof. The present invention further provides a method and composition for treating motor neuron and demyelinating diseases which comprises administering an effective amount of a combination of citicoline and a glucocorticoid, preferably dexamethasone, prednisone or methylprednisolone. The present invention also relates to the use of citicoline for the preparation of a pharmaceutical medicament for the treating of motor neuron and demyelinating diseases, comprising admixing an effective amount of citicoline with a pharmaceutically acceptable carrier.

It is, therefore, one object of the invention to provide methods for improving the-treatment of symptoms of those afflicted with a motor neuron or demyelinating disease.

Another object of the invention is to provide methods for decreasing symptoms in patients who have suffered nerve injury or nerve death due to motor neuron or demyelinating disease.

Still another object of the invention is to provide methods for preventing the worsening of symptoms over the course of the disease, i.e., to inhibit progression.

These and other objects of the invention will be apparent to those of ordinary skill in view of the discussion above and the additional detailed description provided below relating to preferred embodiments of the invention.

### DETAILED DESCRIPTION OF THE PREFERRED EMBODIMENTS

Citicoline is believed to have multiple therapeutic effects. Although the relative contribution of each effect on the treatment of motor neuron or demyelinating disease is unknown, citicoline and its metabolites—which include cytidine and choline—are believed to play important roles in the generation of phospholipids involved in membrane formation and repair. These compounds also are believed to contribute to critical metabolic functions, such as the formation of nucleic acids, and the synthesis of the neurotransmitter acetylcholine. Thus, under conditions of frank neuronal damage with associated nerve cell degeneration, citicoline may function to: (1) stabilize membranes by providing substrates for membrane maintenance; (2) repair damaged membranes by supplying important substrates for membrane formation; and, (3) restore neuronal function by supplying a substrate for the formation of acetylcholine. Moreover, unlike other proposed therapeutic agents, citicoline has the potential not only to stabilize the size or locale of the area of damage, but also to contribute to the repair of the damaged area.

Without being limited by theory, it is believed that citicoline has at least a dual mechanism of action: limiting nerve damage and further progression of disease and aiding in the repair of damaged neuronal tissues. Administration of citicoline is believed to limit the extent of the tissue damage by preventing the accumulation of toxic free fatty acids. In addition, following its administration, it is believed that citicoline is broken down into components, including cytidine and choline, which are substrates required in the formation of phosphatidylcholine, the primary phospholipid of nerve cell membranes, via the Kennedy pathway. It is further postulated that to normalize brain and/or muscle function, nerve cells damaged by motor neuron or demyelinating diseases must manufacture new membrane elements. As described below, in preclinical animal models of ALS and MS, administration of citicoline is shown to reduce the functional deficits produced by nerve degeneration.

Citicoline is preferably administered orally as a pharmaceutically acceptable salt. The preferred salt is the monosodium salt of citicoline, as this form is readily available in pharmaceutically acceptable purity. Citicoline monosodium is an exogenous form of cytidine-5′-diphosphocholine (CDP-choline). Endogenous CDP-choline is a key intermediate in the biosynthesis of membrane phosphatidylcholine, the primary lipid membrane component involved in the dynamic regulation of cellular integrity.

Citicoline may be administered in the following daily dosages. All dosages are provided on a citicoline monosodium basis and on a per patient basis (ranging from about 45 kg to about 100 kg per patient or 70 kg patient on average).

Generally daily citicoline dosages may range from about 100 mg to about 5000 mg, desirably from about 250 to about 3000 mg and preferably from about 500 to about 2000 mg. Doses may be administered once or up to four or more times daily. A highly preferred dosage is 500 mg administered twice per day per patient. If greater therapeutic efficacy is required, a preferred administration is 2000 mg administered in either a single 2000 mg dose or two 1000 mg doses.

The treatment length is variable, but it has been observed that patients tolerate citicoline well at doses ranging from about 250 mg to about 2000 mg for prolonged periods, that is, from several weeks to several years. Dosages may be varied over time depending on the severity of symptoms, individual patient tolerance, route of administration and response to treatment. Treatment may be continued indefinitely if needed and if tolerated well.

Preferably, citicoline is administered orally in the form of capsules, cachets, tablets or lozenges, or as a powder or granules for reconstitution as a solution or suspension in an aqueous or non-aqueous liquid. Administration may also be in the form of a bolus, electuary, suppository, or paste. Formulations for inhalation, or intranasal administration are also contemplated.

Formulations of the active ingredient, suitable for parenteral administration, may comprise a sterile, aqueous preparation of the citicoline active ingredient. The formulations may be presented in unit dosage form and may be prepared by any of the methods well-known in the art of pharmacology.

In addition to containing the standard and well known pharmaceutical carriers and/or excipients, all of the above formulations may contain other therapeutically active substances. Thus, the present invention also contemplates a combination treatment regimen that relates to the co-administration of citicoline and at least a second therapeutic agent, or the respective pharmaceutically acceptable salts thereof.

Broad categories of the at least second therapeutic agent are contemplated. These agents include, but are not limited to, glutamate and glycine antagonists such as neurontin, and drugs such as ACTH, glucocorticoids (e.g., methylprednisolone, prednisone and dexamethasone), antiinflammatory drugs, diphenylhydramine, quinine, myotrophin, or IGF-1, BDNF, BFGF, beta-interferon, Betaseron, Copaxone, Baclofen, Riluzole, and epitopes of myelin basic proteins and the like, which are often used to treat ALS or MS.

Yet other therapeutic agents useful in combination with citicoline are calcium channel blockers (e.g., AJ-394, AK-275, Calpain inhibitors, CD-349, Clentiaze, CNS-1237, CNS-2103, CPC-304 and CPC-317, Dazodipine, Diperdinine, Emopamil, Fasudil, Lacidipine, Lifarizine, Lomerizine, Magnesium, MDL:28170, NB-818, Nilvadipine, Nimodipine, NS-626 and related compounds, SM-6586, SNX-111, S-312-d, U-92032, UK-74505, US-035 and the like), agents targeted at nitric oxide, agents targeted at various other neurotransmitters (e.g., alpha₂-receptor therapeutics, CV-5197, Dopamine receptors, Enadoline, Lazabemide, Milnacipran, Nalmefene, RP-60180, SR-57746A, synaptic uptake blockers and the like), cytokines, hormones and related products (e.g., AN-100225 and AN-100226, Calcitonin gene-related peptides, CEP-075 and related compounds, Ciliary neurotrophic factor, Endothelial cell factor, Endothelin inhibitors, FR-139317 Interleukin-1 receptor antagonist (lipocortin), JTP-2942, Macrophage-regulating compounds, Motoneuronetrophic factor NBI-117, Nerve growth factor, Neural stem cells, Neutrophil inhibitory factor, NS-506, NT-3, Posatirelin, Schwann cell promoters, sCR1, Somatomedin-1 and the like), free radical scavengers (e.g., EPC-K1, MCI-186, Nicaraven, Phenazoviridin, Resorstatin, Rumbrin, Superoxide dismutase, Tirilazad mesylate, U-88999E, Yissum project P-0619, YM-737 and the like), gangliosides and related products (e.g., LIGA4, LIGA4, Monosialoganglioside (GM1), ND-37, Siagoside and the like).

Still other classes of second therapeutic agents include, but are not limited to, modulators of various specific enzymes, neuroprotectives with “diverse” actions (e.g., Ademetionine sulphate tosilate, Ancrod, Apocuanzine, CPC-111, CPC-211, HSV vectors, KF-17329 and KF-19863, LY-178002, MS-153, Nicorandil, N-3393 and N-3398, SUN 4757, TJ-8007, VA-045 and the like, and imaging or contrast agents).

Therefore, a method is provided of treating a subject who is suffering from motor neuron or demyelinating disease comprising co-administering an effective amount of citicoline and at least a second therapeutic agent, or their respective pharmaceutically acceptable salts. The first dose may then be followed by the co-administration of one or more subsequent doses of effective amounts of citicoline alone, the at least second therapeutic agent alone, or their respective pharmaceutically acceptable salts, or as subsequent combinations thereof. Consistent with the other methods disclosed herein, the first dose may be co-administered after diagnosis.. By the use of the term “co-administration,” is meant that the citicoline and the at least second therapeutic agent, or their respective pharmaceutically acceptable salts, are administered together or sequentially.

The method using the contemplated combination therapy includes the administration or co-administration of subsequent doses, which is preferably carried out over a period of at least about 30 days. In a specific embodiment of the invention, the co-administration of subsequent doses is carried out over a period of at least about 4-8 weeks, preferably over a period of at least about six months to about one year. Furthermore, the first dose or subsequent doses are co-administered one or more times daily over the predetermined period. It is anticipated that subjects who may benefit the most from the combination therapy are those who suffer from advanced ALS or other motor neuron disease, or who are in the acute, active stage of a demyelinating disease such as MS, or chronic progressive MS. Maintenance doses may be required for some patients for the rest of their lives.

In the composition, the effective amount of active ingredients in a therapeutic dose may vary according to the particular need. Typical ranges, however, may be from about 100 mg to about 5000 mg of citicoline and about 10 mg to about 1000 mg of at least a second therapeutic agent.

The present invention is illustrated by the Examples that follow, it being understood, however, that the invention is not limited to the specific details of these Examples.

### EXAMPLE 1

### Experimental Allergic Encephalomyelitis (EAE) Model for MS

Experimental allergic encephalomyelitis (EAE) is an inflammatory autoimmune demyelinating disease which can be induced in laboratory animals by injection of myelin basic protein (MBP) or ground spinal cord from another species. This artificially induced disease has become the standard laboratory model for studying clinical and experimental autoimmune diseases. There are many similarities between EAE in animals and MS in humans, including chronic relapse. Thus, EAE is a good predictor of efficacy of drugs and drug combinations for treatment of various autoimmune diseases. Also, because of the similarity in motor symptoms, EAE may also be predictive of drug efficacy for ALS.

The EAE test model is employed to establish the activity of citicoline against MS. Such testing is conducted according to the following procedure.

Thirty female Lewis rats are injected in their foot pads with guinea-pig spinal cord homogenate in complete Freund's adjuvant. The rats are divided into three groups of 10 each. One group is administered citicoline i.p. daily in a dose of 500 mg/kg beginning at 9 days after inoculation. A second group is administered dexamethasone in daily doses of approximately 0.0375 mg/kg s.c., beginning 9 days after inoculation. The third group is a control to which is administered 0.9% saline solution beginning 9 days after inoculation. Duration of the treatment is approximately 17 days. Animals are examined daily, which consists of weighing and scoring for symptoms of EAE according to a disability scale of 0-4.

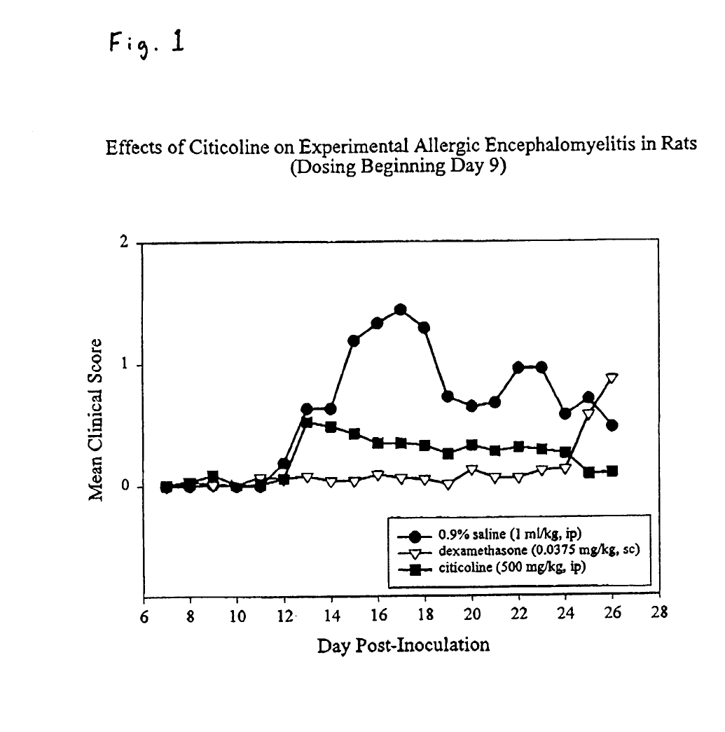

The results of such rat studies are shown in FIG. 1. They establish that citicoline inhibits the progress of EAE, with a dose of 500 mg/kg exhibiting desirable levels of activity. Dexamethasone at a relatively high dosage also inhibits the progress of EAE initially.

### EXAMPLE 2

This example demonstrates the benefits of prevention therapy with citicoline. Thirty-two female Lewis rats are divided into four groups of eight each and on day one administered saline, citicoline (500 or 1000 mg/kg, i.p.), or dexamethasone (0.0375 mg/kg), respectively. Dexamethasone acts as a positive control through its ability to suppress immune function. On day two, experimental autoimmune encephalomyelitis is induced in all rats by injecting ground guinea pig spinal cord in complete Freund's adjuvant in the foot pad. Therapy is continued daily. The rats are tested daily and assigned a functional score of 0 to 4, with 0 being normal and 4 representing death or inability to move. The results, which are summarized in the Table, indicate that administration of citicoline markedly reduces the rapid deterioration of motor function in the test subjects compared to the saline group. Numerical values are averages of the functional scores in each treatment group from Days 14-21 of the study.

| | TABLE | | | | | | | |
|---|---|---|---|---|---|---|---|---|
| | Mean Functional Score | | | | | | | |
| | Day | Day | Day | Day | Day | Day | Day | Day |
| Group | 14 | 15 | 16 | 17 | 18 | 19 | 20 | 21 |
| vehicle | 0.72 | 0.94 | 1.0 | 1.12 | 1.16 | 1.19 | 1.35 | 1.25 |
| (saline) | | | | | | | | |
| Dexamethasone | 0.14 | 0.42 | 0.0 | 0.14 | 0.28 | 0.64 | 0.57 | 0.64 |
| (0.0375 mg/kg) | | | | | | | | |
| citicoline | 0.43 | 0.41 | 0.37 | 0.38 | 0.28 | 0.59 | 0.68 | 0.87 |
| (500 mg/kg) | | | | | | | | |
| citicoline | 0.56 | 0.78 | 0.84 | 0.69 | 0.62 | 0.91 | 0.75 | 0.84 |
| (1000 mg/kg) | | | | | | | | |

### EXAMPLE 3

This example demonstrates the benefits of prevention therapy with a combination of citicoline and dexamethasone. Sixteen female Lewis rats are divided into eight groups of two each and on day one each group is administered one of the following eight solutions:

1. Dexamethasone (Dex) 9.375 μg/kg

2. Dexamethasone 4.6 μg/kg

3. Dexamethasone 2.3 μg/kg

4. Citicoline 500 mg/kg+Dex 9.375 μg/kg

5. Citicoline 500 mg/kg+Dex 4.6 μg/kg

6. Citicoline 500 mg/kg+Dex 2.3 μg/kg

7. Citicoline 500 mg/kg

8. Saline

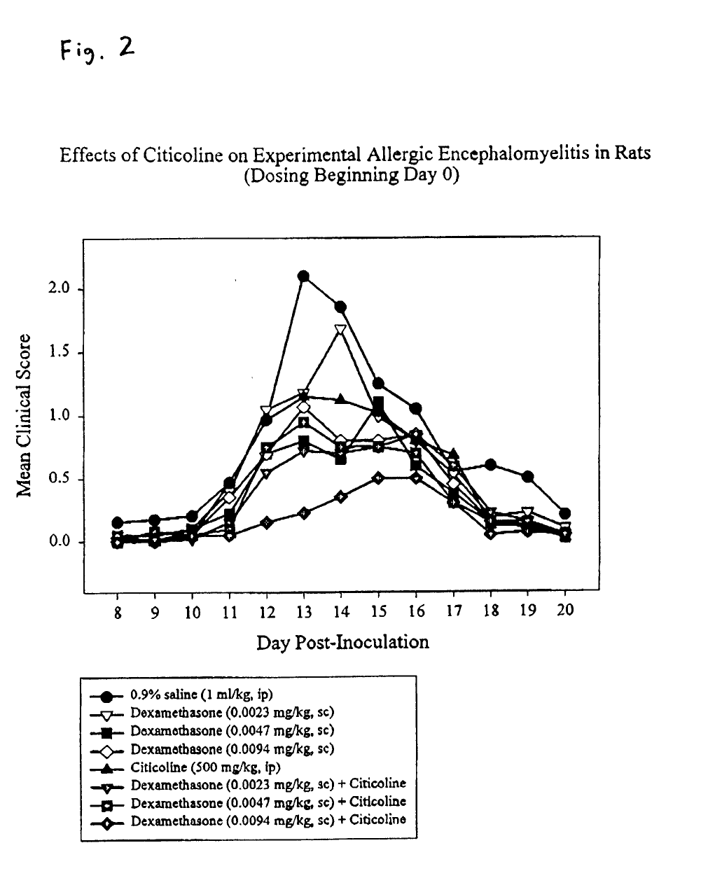

On the same day, experimental autoimmune encephalomyelitis is induced in all rats by injecting ground guinea pig spinal cord in complete Freund's adjuvant in the foot pad. Therapy is continued daily. The rats are tested daily and assigned a functional score of 0 to 4, with 0 being normal and 4 representing death or inability to move. The results, which are summarized in FIG. 2, indicate that administration of citicoline in combination with 2.3 μg/kg of dexamethasone reduces deterioration of motor function in the test subjects to a level equal to or better than that observed for administration of dexamethasone alone at levels up to 9.375 μg/kg. This enhancement of the activity of dexamethasone allows the same results to be obtained without higher doses of dexamethasone, which have the potential for toxic effects, as described in Goodman & Gilman's The Pharmacological Basis of Therapeutics, Ninth Edition (1996) at pages 1474-1476. In addition, administration of citicoline in combination with 9.375 μg/kg of dexamethasone reduces deterioration of motor function substantially compared to the other treatments illustrated in FIG. 2. This increased activity is achieved without exposing the subject to the detrimental effects of higher steroid dosages.

### EXAMPLE 4

This example demonstrates the benefits of post-symptom therapy with a combination of citicoline and dexamethasone. Eight female Lewis rats are divided into four groups of two each and on day one experimental autoimmune encephalomyelitis is induced in all rats by injecting ground guinea pig spinal cord in complete Freund's adjuvant in the foot pad. At symptom onset at approximately day 10, each group is administered one of the following four solutions:

1. Dexamethasone (Dex) 37.5 μg/kg

2. Citicoline 500 mg/kg+Dex 9.375 μg/kg

3. Citicoline 500 mg/kg

4. Saline, 0.9% (1 ml/kg, ip)

Therapy is continued daily. The rats are tested daily and assigned a functional score of 0 to 4, as follows:

0: normal

0.1: tail weaker than normal

0.25: tail fails to curl around examiner's finger

0.75: tail strength only at base

1.0: loss of all tail strength

1.5: limp tail+ failure of one or more hind limbs to grip rotorod

1.5: hind limb dragging (weak movement possible)

1.75: hind limb dragging+ failure of one or more hind limbs to grip rotorod

2.0: hind limb paralysis

3.0: hind limb paralysis+ fail rotorod test

4.0: total paralysis or death

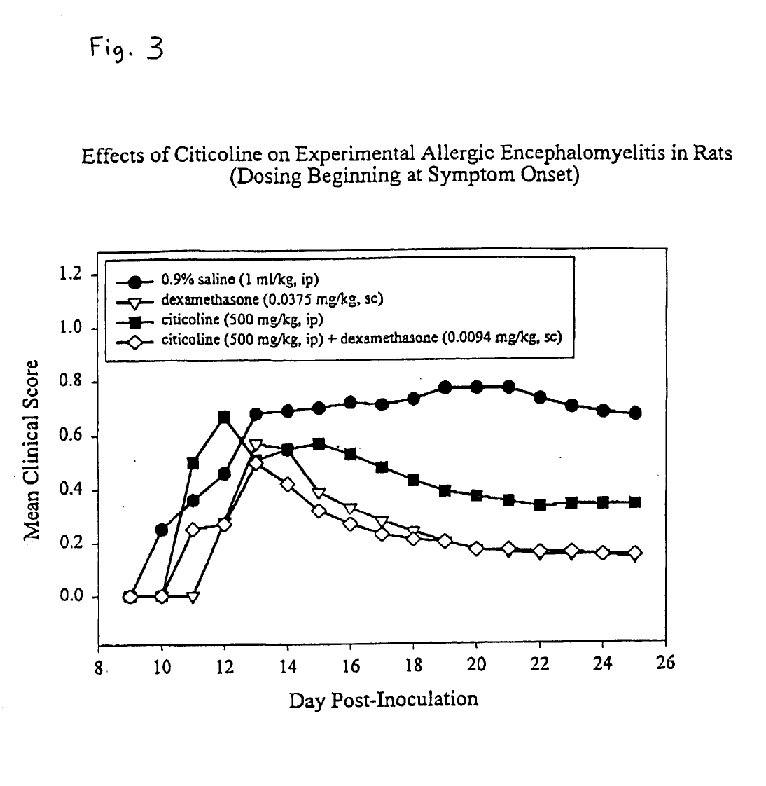

The results, which are summarized in FIG. 3, indicate that administration of citicoline in combination with 9.375 μg/kg of dexamethasone reduces deterioration of motor function in the test subjects to a level equal to or better than that observed for administration of dexamethasone alone at a relatively high level of 37.5 μg/kg. This enhancement of the activity of dexamethasone allows the same results to be obtained without higher doses of dexamethasone in a post-symptom treatment regimen. In addition, administration of citicoline in combination with 9.375 μg/kg of dexamethasone reduces deterioration of motor function substantially compared to administration of citicoline alone. This increased activity is achieved without exposing the subject to the detrimental effects of higher steroid dosages.

### EXAMPLE 5

### ALS Animal Model

Twenty male three month old, transgenic mice expressing familial ALS-linked mutations in the cytoplasmic enzyme SOD1 are divided randomly into two groups of ten mice each: ten animals treated with 500 mg/kg citicoline administered i.p. five days a week and ten control animals treated with water.

The mice show progressive weakness arising from selective motor neuron death, perikaryal proximal axonal swelling, axonal degeneration, and severe skeletal muscle atrophy, all symptoms consistent with familial ALS in humans.

After a trial period of about three months, improvements in the motor behavior of treated animals and enhanced survival over control animals is observed. Generally, stabilization of the debilitating symptoms is achieved. In some cases improvements are highly dramatic over the control group.

### EXAMPLES 6-13—HUMAN STUDIES

### EXAMPLE 6

A study of two sets of four patients each with chronic multiple sclerosis is undertaken. Each patient is first examined for normal hepatic, renal, and bone marrow functioning to establish baseline values. Each of the patients in each group is then treated either with citicoline dissolved in sterile preservative-free isotonic saline 10% or oral tablet or capsule. The citicoline is administered orally or intravenously at a dosage of 250, 500, or 1,000 mg each patient each day for six months. Patients are examined on a daily basis. During the treatment period, daily blood counts and twice weekly blood chemistries are performed on each patient. The neurologic function of each of these patients is measured using the expanded Krutzke disability status scale (EDSS), and the Scripps neurologic rating scale (SNRS).

There is no evidence of any significant toxic side effects. None of the eight patients exhibit any nausea, vomiting, skin rash, or hepatic or renal dysfunction.

In essence, there is no evidence of toxicity in these eight patients with normal marrow, hepatic and renal function. Likewise, the side effects of the citicoline are imperceptible in these eight patients.

Measurement of neurologic function using the EDSS and SNRS scales provides evidence of improvement in MS patients during treatment with citicoline.

### EXAMPLE 7

Six patients with bulbar palsy caused by ALS are treated with 500-1000 mg each citicoline by injection (monosodium salt dissolved in 10 ml sterile isotonic solution of sodium chloride) or by a solid tablet or soft gel capsule orally and administered daily.

As a result of treatment, remarkable improvements in bulbar symptoms are observed, all six patients (three in each group) being able to speak after 21 days of treatment. The side effects of this treatment are insignificant and can be neglected in consideration of the improvement obtained. Treatment is continued for 60 days and all six patients exhibit improvement for several days after treatment is halted.

### EXAMPLE 8

A double-blind study against placebo is performed as follows: 77 patients suffering from ALS are treated with 500 mg each citicoline by injection (monosodium salt dissolved in 10 ml sterile isotonic solution of sodium chloride) or be a solid tablet or soft gel capsule orally and administered daily, for a period of 12 to 18 months, and 78 patients receive a placebo.

The results obtained are analyzed in terms of survival in the study, it being understood that “study drop-outs” (departures from the study) include individuals who actually die, and also individuals whose clinical state necessitates a tracheotomy or transfer to assisted ventilation.

In this study, 50% of patients on placebo die, whereas this percentage drops to 20% in patients on citicoline.

The probability in Wilcoxon's test (R. L. PREUCTICE, Bioimetrika, 65, 167-179 (1978)) is equal to 0.02 and the probability in the stratified log-rank test (R. PETO and J. PETO, Journal of the Royal Statistical Society, series A, vol. 135, 185-207 (1972)) is equal to 0.09.

In subjects suffering from ALS with early bulbar involvement or the bulbar form of the disease (the most serious form of the disease; the usual mean survival of this type of patient is less than 3 years), 65% of patients on placebo die, whereas this percentage drops to 30% in patients on citicoline. The probability in Wilcoxon's test is equal to 0.020 and the probability in the log-rank test is equal to 0.047.

### EXAMPLE 9

Six patients ranging in age from 33 to 38 who have suffered from multiple sclerosis for more than 6 years and have been bedridden for at least 4 months are treated with steroids after which walking is possible with the aid of a walker. A wheelchair is needed for travel outside the home. The attending physician advises the patients that without chemotherapy (including treatment with cytoxan), the patients would return to the bedfast state within 6 months.

The patients elect to discontinue all prescribed therapy and begin taking approximately 1000 mg oral citicoline daily. The patients observe no change in the status of the disease until about 10 weeks have elapsed at which time they report feeling better than at any time in the previous year. Within two to four additional weeks, all patients are able to walk longer distances.

### EXAMPLE 10

Four patients with a history of leg weakness, progressive loss of voice and multiple other neuromuscular symptoms are diagnosed with ALS. The patients start on 250 mg/day oral citicoline and an extensive exercise program.

Three months after therapy initiation, the condition of all six patients deteriorates slightly, including progressive weakening of the voice. However, the worsening of condition is minimal compared to baseline ALS progression.

### EXAMPLE 11

Five patients ranging in age from 31 to 47 are diagnosed with Charcot-Marie Tooth disease. The diagnosis is confirmed by genetic studies revealing a duplicate locus on chromosome 17p11.2 containing a gene for a peripheral myelin protein. All five patients exhibit weakness and wasting of skeletal muscles as well as sensory changes. Three are bedridden while the other two are confined to wheelchairs.

The patients discontinue all prescribed therapy and begin taking 750 mg oral citicoline daily. No improvement is observed for 12 weeks, after which progressive improvement is observed in each case. After an additional four weeks of therapy, the bedridden patients are able to use a wheelchair while the previously wheelchair bound patients can use a walker for short distances.

### EXAMPLE 12

Seven patients ranging in age from 12 to 22 are diagnosed with Friedreich's ataxia. All patients exhibit pes cavus (foreshortening of the feet) with cocking of the toes and unsteadiness in walking. The two oldest patients also exhibit dysarthria.

The patients discontinue all prescribed therapy and begin taking 500 mg oral citicoline daily. After four weeks, progressive improvement is observed in each case. All patients exhibit greater stability in walking and the two oldest patients show improvement in speech.

### EXAMPLE 13

A. H. is a 54 year-old white, right-handed, married female who presents with chronic progressive multiple sclerosis. The patient reports first known neurological event to have occurred at 28 years old. She described an optic neuritis event of her left eyes. She was told at that time that she could have one of a variety of medical conditions, including multiple sclerosis. She was given prednisone by a general practitioner, and her optic neuritis syndrome appeared to have resolved. She remained symptom-free for approximately ten years.

At age 38, she described the recurrence of optic neuritis. She was more descriptive of that event where the vision in the center of her eye appeared distorted. Peripheral vision was apparently unaffected. Initially, she pursued the care of an ophthalmologist who felt the etiology was once again related possibly to multiple sclerosis, She went on to pursue further evaluation of an internist who later referred her to a neurologist. At the time, she was described as having relapsing-remitting multiple sclerosis. She received a course of ACTH therapy. Initially, the symptoms had resolved, but she went on to describe exacerbations of ophthalmological symptoms between 1982 and 1996, alternating between the right and left eyes. In 1986, she described the onset of “falls.” She recalls being affected by the dragging of her left foot on occasion and “stubbing her toe frequently” on the ground. Her right lower extremity was notably stronger than the left. She continued to have a gradual decline from that point on and had no further symptom-free periods.

Since around 1986, she has received the following course of treatments and therapies:

Prednisone (worked initially),

Methylprednisolone (marginal effects),

ACTH (worked initially),

Imuran (no effect),

Cytoxan (uncertain of effect as course of treatment was discontinued due to adverse events),

Betaseron (no effect),

Cladribine (completed in January of 1998, and felt to have had no effect),

Non-allergenic diet (avoidance of dairy products and other foods—no effect),

Bee venom therapy (felt to have increased the strength in legs, through this subsided after 3-4 months)

Currently, she is diagnosed with chronic-progressive multiple sclerosis. She is afflicted with visual impairments, chronic fatigue, difficulty with speech articulation, tremors in the right and left hands, decreased fine motor movement in the right greater than left hands and decreased motor strength in her lower extremities. She is without weight bearing and is confined to a wheelchair. When asked what her major barriers are as far as functional abilities on a day to day basis, she places fatigue at the top of her impairment list followed by speech difficulties and decreased fine motor movement of her hands. She experienced marked spasticity of her lower extremities with the progression of her neurological condition over the course of several years and eventually resorted to a baclofen pump which was implanted in 1997. She has had continuous infusions into her spinal fluid since this time, which has reduced the spasticity considerably in her lower extremities. She receives 85 micrograms of baclofen per day. While the baclofen has ameliorated the spasticity, it is felt that this treatment has lessened her ability to bear weight on her lower extremities.

### Citicoline Response

Apr. 5, 1998—The patient started on citicoline. She began her treatment at 500 mg per day every morning for the first two weeks. She reported no adverse side effects during this time as well as no notable therapeutic effects. She and her husband maintained a diary to record medication, potential adverse event and neurological changes.

Apr. 22, 1998—Dosage was increased to 1000 mg per day (500 mg in the a.m. and 500 mg in the p.m.). On the first two days of increased dosage, the patient noted that her balance had been poor and that her fatigue had increased in the early hours of the morning (not her usual time for worsening of fatigue during the day). After two days, however, the patient experienced increased endurance to stay up later into the evenings (normal bedtime was previously 10 p.m.; since the increased dose on citicoline, the patient found herself retiring between midnight and 1:00 a.m.). She was without those early morning fatigue episodes noted on the first few days of therapy, and, in general, was feeling relatively well.

Previously, she was discouraged from using the telephone in that her speech articulation had been poor and perhaps her motivation use the phone had lessened. However, during this period of time, she also found herself both willing and able to conduct telephone inquiries such as to conduct banking activities. She described having a complicated discrepancy with the bank and felt both motivated and capable of articulating the situation to the bank successfully.

May 11, 1998—Dosage was increased to 2000 mg per day (1000 mg in the a.m., 1000 mg in p.m.) Once again, the patient noticed worsening of fatigue in the morning hours for approximately two days, then returned back to her baseline state of fatigue (generally less fatigue in the morning and a gradual increase in fatigue as the day processed). Once again, it was notable on citicoline (2000 mg/day) that the patient was able to stay up for longer periods during the day. She made a notation in a personal diary that on May 14th she was making several phone calls and described an overall feeling of wellness.

Jun. 5, 1998—The patient left for a 4 hour car drive to an out-of-state family function. She recalled an inability to endure long car rides for quite some time, and has avoided talking a car trip of this duration in the past five years due to her subsequent exhaustion following these events. She felt well upon arrival despite the long drive. On June 6th, she forgot to take her citicoline dose throughout the day. She described feeling “so-so and not as well as the day before.” She also missed taking her citicoline dose on the morning of June 7th. She detected a marked change in her physical well-being in the sense that she was not as “up” and attributed this to the return drive back to her home. They arrived back at her house at approximately 2:30 p.m. The patient described noticing a pronounced difference in her overall state with mainly increased fatigue. She described her tremors being worse, making eating more difficult and she felt, in general, that she had to be more careful as her motor and visual judgment had been off. She took her evening dosage of citicoline (1000 mg) and on the following morning she felt that she was back to baseline while on the drug.

At the end of June, A. H. took a cruise to Alaska with her family, enduring the flight to and from the west coast. She tolerated the trip and extensive traveling quite well.

Her neurologist has no knowledge that she is currently taking citicoline. Prior to taking this medication, she was last seen by her neurologist on Apr. 21, 1998. She was seen recently on Jul. 7, 1998. The neurologist reported noticing an improvement in the patient's eyes in that the “eye movement had improved.”. The neurologist also noticed that the strength with hand resistance had improved as well. The patient continues to be on citicoline at a dose of 2000 mg per day.

We claim:

1. A method of treating a motor neuron disease or a demyelinating disease comprising administering a first dose of an effective amount of citicoline or a pharmaceutically acceptable salt thereof.

2. A method of reducing nerve degeneration in a patient with a motor neuron disease or a demyelinating disease comprising administering a first dose of an effective amount of citicoline or a pharmaceutically acceptable salt thereof, followed by chronic administration of subsequent doses of effective amounts of citicoline or a pharmaceutically acceptable salt thereof.

3. A method of treating a patient having a motor neuron disease or a demyelinating disease comprising administering a first dose of an effective amount of citicoline or a pharmaceutically acceptable salt thereof, followed by chronic administration of subsequent doses of effective amounts of citicoline or a pharmaceutically acceptable salt thereof.

4. The method of claim 3, wherein the method comprises treating a patient having ALS or MS.

5. The method of claim 3 wherein said administration of subsequent doses is carried out over a period of at least about 30 days.

6. The method of claim 3 wherein said administration of subsequent doses is carried out over a period of at least about 4-8 weeks.

7. The method of claim 3 wherein said administration of subsequent doses is carried out over a period of at least about six months to about one year.

8. The method of claim 7 wherein said first dose or subsequent doses is administered twice daily over said period.

9. The method of claim 3 wherein said first dose or subsequent doses is administered chronically one or more times daily.

10. The method of claim 3 wherein the subject is a human.

11. A method of treating a patient having a motor neuron disease or a demyelinating disease comprising co-administering a first dose of an effective amount of citicoline or a pharmaceutically effective salt thereof with at least a second therapeutic agent, or its pharmaceutically acceptable salt.

12. The method of claim 11, further including the step of administering at least one subsequent dose of an effective amount of citicoline or a pharmaceutically acceptable salt thereof, at least one subsequent dose of an effective amount of said at least a second therapeutic agent or a pharmaceutically acceptable salt thereof, or both said citicoline and said at least second therapeutic agent, or pharmaceutically acceptable salts thereof.

13. The method of claim 11 wherein said second therapeutic agent inhibits nerve cell degeneration or promotes nerve cell growth.

14. The method of claim 11 wherein said coadministration comprises administering the effective amounts of said citicoline and said at least second therapeutic agent, or their respective pharmaceutically acceptable salts, together or sequentially.

15. The method of claim 11, wherein the method comprises treating a patient having ALS or MS.

16. The method of claim 11 wherein said co-administration of subsequent doses is carried out over a period of at least about 30 days.

17. The method of claim 11 wherein said co-administration of subsequent doses is carried out over a period of at least about 4-8 weeks.

18. The method of claim 11 wherein said co-administration of subsequent doses is carried out over a period of at least about six months to about one year.

19. The method of claim 11 wherein said first dose or subsequent doses is co-administered chronically one or more times daily.

20. The method of claim 11 wherein said first dose or subsequent doses is co-administered twice daily over said period.

21. The method of claim 13 wherein said nerve cell degeneration occurs in the brain or spinal cord.

22. The method-of claim 11 wherein the subject is a human.

23. The method of claim 11 wherein the subject has suffered a potentially debilitating degree of nerve degeneration or nerve cell death.

24. The method of claim 11 wherein said at least a second therapeutic agent is a glucocorticoid.

25. The method of claim 24 wherein said glucocorticoid is dexamethasone, prednisone or methylprednisolone, or their pharmaceutically acceptable esters and phosphates.

26. A composition for the treatment of a patient having a motor neuron disease or a demyelinating disease comprising an effective amount of citicoline and at least a second therapeutic agent, for inhibiting nerve cell degeneration or improving function, or their respective pharmaceutically acceptable salts, in a pharmaceutically acceptable carrier.

27. The composition of claim 26 wherein said effective amount ranges from about 100 mg to about 5000 mg of citicoline per unit dosage and about 10 mg to about 1000 mg of said at least a second therapeutic agent per unit dosage.

28. The composition of claim 27 wherein said at least a second therapeutic agent is a glucocorticoid.

29. The composition of claim 28 wherein said glucocorticoid is dexamethasone, prednisone or methylprednisolone, or their pharmaceutically acceptable esters and phosphates.
